(19) United States
(12) Patent Application Publication
(10) Pub. No.: US 2018/0256611 A1
(43) Pub. Date: Sep. 13, 2018

(54) BISPHOSPHONATES FOR INCREASING THE TYPE 2 CHARACTER OF A MODIC CHANGE

(71) Applicant: ANTECIP BIOVENTURES II LLC, NEW YORK, NY (US)

(72) Inventors: Herriot Tabuteau, New York, NY (US); Jaro Karppinen, Oulu (FI)

(21) Appl. No.: 15/977,413

(22) Filed: May 11, 2018

Related U.S. Application Data
(63) Continuation-in-part of application No. PCT/US18/13625, filed on Jan. 12, 2018.
(60) Provisional application No. 62/445,646, filed on Jan. 12, 2017.

(51) Int. Cl. A61K 31/675; A61P 25/04; A61K 9/00

(52) U.S. Cl. CPC A61K 31/675; A61K 9/0053; A61P 25/04

(57) ABSTRACT

Oral dosage forms of bisphosphonates, such as zoledronic acid, in an acid form or a salt form can be used to treat or alleviate pain or related conditions. Zoledronic acid also tends to speed up the conversion of M1-dominant to M2-dominant Modic changes and decrease the volume of M1-dominant Modic changes, which correlate with improvement in symptoms.

### CROSS-REFERENCE TO RELATED APPLICATIONS

This application is a continuation-in-part of international Pat. App. No. PCT/US18/13625, filed Jan. 12, 2018, which claims the benefit of U.S. provisional Pat. App. No. 62/445,646, filed Jan. 12, 2017, both of which are incorporated by reference in their entireties.

### BACKGROUND

Bisphosphonate compounds are potent inhibitors of osteoclast activity, and are used clinically to treat bone-related conditions such as osteoporosis, Paget's disease of bone, and cancer-related conditions including multiple myeloma and bone metastases from solid tumors. Bisphosphonates generally have low oral bioavailability.

Patchy osteoporosis and bone marrow edema may result from osteoclast hyperactivity. Zoledronic acid is a potent inhibitor of bone resorption and osteoclast activity. Nitrogen containing bisphosphonates, such as zoledronic acid, also inhibit the mevalonate pathway in the osteoclast thereby interrupting normal osteoclast function.

### SUMMARY

This disclosure relates to administration of a bisphosphonate, such as zoledronic acid (e.g. oral zoledronic acid or intravenous zoledronic acid), which results in an increase in the Modic change type 2 (M2) character of a modic change (MC) having at least some Modic change type 1 (M1) character.

MC are more common among patients with low back pain (LBP) than in asymptomatic volunteers. M1 may also be associated with more painful or severe LBP than other types of MC. Conversion of M1 to M2 may be associated with improvement, or decrease, of pain intensity and disability.

In some embodiments, zoledronic acid (e.g. oral zoledronic acid or intravenous zoledronic acid) has a tendency toward conversion from M1-dominant to M2-dominant and M1 volume tends to decrease.

In some embodiments, zoledronic acid (e.g. oral zoledronic acid or intravenous zoledronic) tends to speed up the conversion of M1-dominant to M2-dominant MC and decrease the volume of M1-dominant MC, which correlates with improvement in symptoms.

### BRIEF DESCRIPTION OF DRAWINGS

FIG. 1 depicts the plasma concentration of zoledronic acid in dogs over time after administration of 150 mg of the disodium salt form of zoledronic acid and the diacid form of zoledronic acid.

FIG. 2 depicts the course of MC-types of primary MC during one-year follow-up. Arrows indicate the change of MC-type (in percent).

FIG. 3 depicts scatter plots showing A) the positive correlation between change in M1 volume and change in low back pain intensity and B) the negative correlation between change in M2 volume and change in low back pain intensity.

FIG. 4 depicts scatter plots showing A) the positive correlation between change in M1 volume and change in Oswestry Disability Index and B) the negative correlation between change in M2 volume and change in Oswestry Disability Index.

### DETAILED DESCRIPTION

Bisphosphonates may be used to increase the M2 character of a mixed M1/M2 Modic change or an M1 Modic change. This may help to reduce the pain suffered by the patient, particularly a patient suffering from low back pain.

Modic changes (MC) are vertebral bone marrow changes adjacent to the endplates on magnetic resonance imaging (MRI). Lumbar MC are associated with low back pain (LBP) and preliminary data indicates that persistence of type 1 MC (M1) is associated with persistence of symptoms. Three different types of MC have been described; Type 1 MC show fibrovascular replacement of bone marrow and are considered to be the earliest stage in the process of the evolution of MC, representing an inflammatory lesion; Type 2 MC (M2) show fatty replacement of the red bone marrow; while Type 3 MC (M3) are associated with subchondral bone sclerosis. The identification of mixed types MC (M1/2 and M2/3) is thought to indicate different stages of the same pathologic process, as MC can convert from one type into another.

Any suitable bisphosphonate may be used, such as pamidronate or pamidronic acid, neridronate or neridronic acid, olpadronate or olpadronic acid, alendronate or alendronic acid, incadronate or incadronic acid, ibandronate or ibandronic acid, risedronate or risedronic acid, cimadronate or cimadronic acid, zoledronate or zoledronic acid, etidronate or etidronic acid, clodronate or clodronic acid, tiludronate or tiludronic acid, etc. In some embodiments, the bisphosphonate is zoledronic acid.

Zoledronic acid or another bisphosphonate may be administered to a human patient in a variety of forms adapted to the chosen route of administration, e.g., orally, rectally, or parenterally. Parenteral administration in this respect includes, but is not limited to, administration by the following routes: pulmonary, intrathecal, intravenous, intramuscular, subcutaneous, intraocular, intrasynovial, transepithelial including transdermal, sublingual and buccal; topically; nasal inhalation via insufflation; and rectal systemic. In some embodiments, zoledronic acid is administered orally.

Unless otherwise indicated, any reference to a compound herein, such as zoledronic acid, by structure, name, or any other means, includes pharmaceutically acceptable salts, such as the disodium salt; alternate solid forms, such as polymorphs, solvates, hydrates, etc.; tautomers; or any other chemical species that may rapidly convert to a compound described herein under conditions in which the compounds are used as described herein. Unless otherwise indicated, a phrase such as “administering a bisphosphonate,” “administering zoledronic acid,” includes administering any form of the bisphosphonate, zoledronic acid, etc., such as those recited above.

In some embodiments, zoledronic acid is administered in a dosage form comprising a salt form, such as a salt of a dianion of zoledronic acid. In some embodiments, zoledronic acid is administered in a dosage form comprising a disodium salt form of zoledronic acid. In some embodiments, zoledronic acid is administered in a sodium salt form, such as a monosodium salt, a disodium salt, a trisodium salt, etc. In some circumstances, use of the disodium salt may be desirable. For example, the disodium salt is much more soluble in water than the diacid form. As a result, in some processes, the disodium salt can be easier to work with than the diacid form. Additionally, the sodium salt, such as the disodium salt, may be more bioavailable and/or more rapidly absorbed when taken orally as compared to the diacid form.

The term “treating” or “treatment” broadly includes any kind of treatment activity, including the diagnosis, cure, mitigation, or prevention of disease in human or other animals, or any activity that otherwise affects the structure or any function of the body of human or other animals.

Commonly used measures of pain intensity include the visual analog scale (VAS) and the numerical rating scale (NRS). With the VAS approach, patients rate the severity of their pain by marking a point on a 10-cm (or 100 mm) VAS (0=no pain and 10=worst possible pain). With the NRS approach, patients rate the severity of their pain by verbally responding to a 10-point NRS (0=no pain and 10=worst possible pain). VAS and NRS scores have been shown to be strongly correlated (slope of regression line, 1.01), indicating that a score on the 10-cm VAS is equivalent to the same score on 10-point NRS (Bijur P E et al. Acad Emerg Med 2003; 10:390-392). For example, a VAS score of 5 cm (or 50 mm) is equivalent to an NRS score of 5. Pain in a person with a VAS score of 5 cm or 50 mm or higher, or an NRS score of 5 or higher, may be referred to herein as moderate to severe pain.

Some patients with more severe pain may respond better to treatment as compared to other patients with less severe pain. In some embodiments, the person has baseline pain intensity of 4 or greater measured using the 0-10 numerical rating scale (NRS), or 4 cm or greater using the 10 cm visual analog scale (VAS).

In some embodiment, the person being treated to increase the M2 character of a Modic change has an NRS of 5 or greater, or a VAS of 5 cm or greater. In some embodiments, the patient has an NRS of 4 or greater, or a VAS of 4 cm or greater. In some embodiments, the patient has an NRS of 6 or greater, or a VAS of 6 cm or greater. In some embodiments, the patient has an NRS of 7 or greater, or a VAS of 7 cm or greater. In some embodiments, the patient has an NRS of about 1, about 2, about 3, about 4, about 5, about 6, about 7, about 8, about 9, or about 10. In some embodiments, the patient has a VAS of about 1 cm, about 2 cm, about 3 cm, about 4 cm, about 5 cm, about 6 cm, about 7 cm, about 8 cm, about 9 cm, or about 10 cm. In some embodiments, the patient has a severe pain with an NRS of 7 or greater, or a VAS of 7 cm or greater.

For some patients with low back pain or pain associated with Modic changes, treatment with a nitrogen-containing bisphosphonate, such as zoledronic acid (e.g. oral zoledronic acid), to increase the M2 character of a Modic change may decrease the visual analog (VAS) pain score using a 10 cm scale, by at least about 0.5 cm, at least about 0.8 cm, at least about 1 cm, at least about 1.5 cm, up to about 5 cm, or up to about 10 cm, as compared to a placebo.

For some patients with low back pain or pain associated with Modic changes, treatment with a nitrogen-containing bisphosphonate, such as zoledronic acid (e.g. oral zoledronic acid), to increase the M2 character of a Modic change may decrease the numerical rating scale (NRS) pain score using a 0-10 scale, by at least about 0.1, at least about 0.5, at least about 0.8, at least about 1, at least about 1.5, up to about 5, or up to about 10. In some embodiments, the NRS score may be decreased by at least about 0.1, at least about 0.5, at least about 0.8, at least about 1, at least about 1.5, up to about 5, or up to about 10, as compared to a placebo.

Increasing the M2 character of a Modic change, such as by converting M1 to M2 or M1/M2, or by converting an M1 dominant M1/M2 to an M2 dominant M1/M2, may result in long term reduction in pain, such as a reduction in pain that lasts at least about one month, about two months, about three months, about four months, about six months, about twelve months, about 2 years, or longer. According to some embodiments, administration of a bisphosphonate, such as zoledronic acid, achieves a reduction in pain for greater than three hours with a duration of no more than about three months, no more than about four months, no more than about five months, or no more than about six months.

According to some embodiments, after administration of a bisphosphonate, such as zoledronic acid (e.g. oral zoledronic acid), an increase in the M2 character of a Modic change relative to the M2 character prior to administration may be observed for up to three months, four months, five months, six months, twelve months, 2 years, or longer. According to some embodiments, after the administration of a bisphosphonate, such as zoledronic acid, the size of Modic changes relative to the size prior to administration is reduced at about three months, at about four months, at about five months, at about six months, or at about twelve months.

A Modic change that has increased M2 character may be found in the cervical, thoracic, lumbar, or sacral spine. Modic changes may be found at various spinal levels such as at C1/2, C2/3, C3/4, C4/5, C5/6, C6/7, C7/T1, T1/2, T2/3, T3/4, T4/5, T5/6, T6/7, T7/8, T8/9, T9/10, T10/11, T11/12, T12/L1, L1/2, L2/3, L3/4, L4/5, or L5/S1, etc., any of which may be treated using a bisphosphonate, such as zoledronic acid.

In some embodiments, the Modic change being treated is located at L2/3. In some embodiments, the Modic change being treated is located at L3/4. In some embodiments, the Modic change being treated is located at L4/5. In some embodiments, the Modic change being treated is located at L5/S1.

In some embodiments, the Modic change being treated is located at C3/4. In some embodiments, the Modic change being treated is located at C4/5. In some embodiments, the Modic change being treated is located at C5/6. In some embodiments, the Modic change being treated is located at C6/7.

In some embodiments, the Modic change being treated is located at T5/6. In some embodiments, the Modic change being treated is located at T6/7. In some embodiments, the Modic change being treated is located at T7/8. In some embodiments, the Modic change being treated is located at T8/9. In some embodiments, the Modic change being treated is located at T9/10.

In some embodiments, the patient being treated has Modic changes at two or more levels. In some embodiments, the patient being treated has Modic changes at three or more levels. In some embodiments, greater pain relief is obtained when treating a patient with Modic changes at two, three, or more levels, than treating a patient with Modic changes at a single level.

In some embodiments, greater pain relief is obtained when treating a patient with Modic changes at two levels than treating a patient with Modic changes at a single level.

In some embodiments, greater pain relief is obtained when treating a patient with Modic changes at three or more levels than treating a patient with Modic changes at a single level.

In some embodiments, greater pain relief is obtained when treating a patient with Modic changes three or more levels than treating a patient with Modic changes at two levels.

In some embodiments, the administration of a nitrogen-containing bisphosphonate, including e.g. zoledronic acid, to a patient or mammal in need thereof, achieves a reduction in the size of Modic changes relative to baseline, such as M1 Modic changes, of at least about 5%, at least about 10%, at least about 15%, at least about 20%, at least about 25%, at least about 30%, at least about 40%, at least about 50%, at least about 60%, at least about 70%, at least about 80%, at least about 90%, or about 100%. In some embodiments, the reduction in the size of Modic changes represents an improvement of the treatment relative to placebo of at least about 10%, at least about 15%, at least about 20%, at least about 25%, at least about 30%, at least about 40%, at least about 50%, at least about 60%, at least about 70%, at least about 80%, at least about 90%, at least about 100%, at least about 120%, at least about 150%, at least about 170%, at least about 200%, at least about 250%, at least about 300%, at least about 350%, at least about 400%, or at least about 450%. In some embodiments, the use of a bisphosphonate inhibits an increase in the size of Modic changes over time.

The oral bioavailability of a bisphosphonate in a dosage form can vary. Some dosage forms may have ingredients added to enhance the bioavailability. However, bioavailability enhancement is not necessary for an oral dosage form to be effective. In some embodiments, the dosage form is substantially free of bioavailability-enhancing agents, such as amino acids or large quantities of carboxylic acid salts (e.g. the dosage form contains less than about 1%, 5%, 10%, 20%, 50%, or 70% enhancer, such as an amimo acid or a carboxylic acid salt enhancer, by weight).

The oral bioavailability of zoledronic acid may be enhanced by orally administering the zoledronic acid in a salt form, such as a disodium salt form.

In some embodiments, a disodium salt form of zoledronic acid provides an enhancement to bioavailability, as compared to the diacid form of zoledronic acid, which adds to any enhancement to bioavailability provided by any bioavailability-enhancing agents in the dosage form. In some embodiments, the disodium salt form of zoledronic acid provides an enhancement to bioavailability, as compared to the diacid form of zoledronic acid, which is greater than any enhancement to bioavailability provided by any bioavailability-enhancing agents in the dosage form. In some embodiments, the disodium salt form of zoledronic acid may be administered in a dosage form that is substantially free of bioavailability-enhancing agents. In some embodiments, the disodium salt form of zoledronic acid may be administered in a dosage form that is free of bioavailability-enhancing agents.

For example, the bioavailability of zoledronic acid in a disodium salt form may be improved by at least about 10%, at least about 20%, at least about 30%, at least about 50%, and/or up to about 100%, or up to about 200%, as compared to administration of zoledronic acid in the diacid form. For example, a disodium salt form of zoledronic acid may increase the oral bioavailability of the diacid form of zoledronic acid from about 0.8-1% for the diacid form, to about 1.2-3% for the disodium salt form without using bioavailability enhancers such as fatty acid salts or amino acids, e.g. about 1.2-1.4%, about 1.3-1.5%, about 1.4-1.6%, about 1.5-1.7%, about 1.6-1.8%, about 1.7-1.9%, about 1.8-2%, about 1.9-2.1%, about 2-2.2%, about 2.1-2.3%, about 2.2-2.4%, about 2.3-2.5%, about 2.6-2.8%, about 2.7-2.9%, about 2.8-3%, about 1.2-1.5%, about 1.5-2%, about 1.7-2.3%, or about 2-3%, etc. In addition to the disodium salt form, other forms of zoledronic acid may have a bioavailability, without bioavailability enhancement, that is about 1.2-3%, e.g. about 1.1-1.3%, about 1.2-1.4%, about 1.3-1.5%, about 1.4-1.6%, about 1.5-1.7%, about 1.6-1.8%, about 1.7-1.9%, about 1.8-2%, about 1.9-2.1%, about 2-2.2%, about 2.1-2.3%, about 2.2-2.4%, about 2.3-2.5%, about 2.6-2.8%, about 2.7-2.9%, about 2.8-3%, about 1.2-1.5%, about 1.5-2%, about 1.7-2.3%, or about 2-3%, etc. These bioavailabilities may also be observed in dosage forms containing bioavailability enhancers such as fatty acid salts or amino acids. For example, other forms of zoledronic acid with lower bioavailability may be combined with enhancers to obtain dosage forms that have the bioavailablity values in these ranges. Additionally, lower amounts of enhancers, or less effective enhancers, might result in dosage forms having bioavailability values within these ranges.

Some oral dosage forms comprising zoledronic acid have a dose of zoledronic acid and a configuration suitable for a particular species of mammal, e.g. dog, rat, human, etc. Such a dosage form may have zoledronic acid present in an amount that results in a desired range for an area under the plasma concentration curve (AUC) of zoledronic acid in that particular species of mammal. For example the dose of zoledronic acid and a configuration of the oral dosage form may result in an AUC of zoledronic acid in human beings of about 1 ng·h/mL to about 700 ng·h/mL, about 3 ng·h/mL to about 30 ng·h/mL, about 3 ng·h/mL to about 10 ng·h/mL, about 50 ng·h/mL to about 700 ng·h/mL, about 130 ng·h/mL to about 180 ng·h/mL, about 300 ng·h/mL to about 450 ng·h/mL, about 300 ng·h/mL to about 350 ng·h/mL, about 300 ng·h/mL to about 310 ng·h/mL, about 340 ng·h/mL to about 350 ng·h/mL, about 370 ng·h/mL to about 420 ng·h/mL, about 380 ng·h/mL to about 390 ng·h/mL, about 405 ng·h/mL to about 415 ng·h/mL, about 140 ng·h/mL to about 160 ng·h/mL, about 140 ng·h/mL to about 150 ng·h/mL, about 150 ng·h/mL to about 160 ng·h/mL, about 140 ng·h/mL, 142 ng·h/mL, about 155 ng·h/mL, about 305 ng·h/mL, 304 ng·h/mL, about 345 ng·h/mL, 343 ng·h/mL, about 385 ng·h/mL, 384 ng·h/mL, about 410 ng·h/mL, or any AUC in a range bounded by, or between, any of these values, upon administration of the oral dosage form to a mammal.

Unless otherwise indicated, the AUC refers to the AUC calculated to the last measured concentration (AUC₍₀₋ₜ₎) and extrapolated to infinity (AUC_{(0-inf)}).

An oral dosage form comprising zoledronic acid having a dose of zoledronic acid and a configuration suitable for a particular species of mammal may have zoledronic acid present in an amount that results in a Cₘₐₓ of zoledronic acid of about 0.2 ng/mL to about 300 ng/mL, about 0.5 ng/mL to about 5 ng/mL, about 5 ng/mL to about 300 ng/mL, about 5 ng/mL to about 50 ng/mL, about 20 ng/mL to about 50 ng/mL, about 30 ng/mL to about 50 ng/mL, about 50 ng/mL to about 200 ng/mL, about 50 ng/mL to about 150 ng/mL, about 80 ng/mL to about 120 ng/mL, about 90 ng/mL to about 100 ng/mL, about 50 ng/mL to about 200 ng/mL, about 40 ng/mL, about 95 ng/mL, about 97 ng/mL, or any Cₘₐₓ in a range bounded by, or between, any of these values, upon administration of the oral dosage form to a mammal.

An oral dosage form comprising zoledronic acid having a dose of zoledronic acid and a configuration suitable for a particular species of mammal may be configured so that administration of the oral dosage form to the particular species of mammal results in a Tₘₐₓ of zoledronic acid of about 0.4 hr to about 1 hr, about 0.5 hr, or about 0.75 hr, or any Tₘₐₓ in a range bounded by, or between, any of these values.

In some embodiments, the zoledronic acid in the disodium salt form is present in an amount such that the oral dosage form provides an area under the plasma concentration curve of zoledronic acid of about 4 ng·h/mL to about 2000 ng·h/mL to the mammal each time the zoledronic acid in the disodium salt is administered.

In some embodiments, the zoledronic acid, including zoledronic acid in an acid form or a salt form, e.g the disodium salt form, is present in an amount such that the oral dosage form provides an area under the plasma concentration curve of zoledronic acid of about 100 ng·h/mL to about 2000 ng·h/mL, about 100 ng·h/mL to about 1000 ng·h/mL, about 500 ng·h/mL to about 1000 ng·h/mL, or about 500 ng·h/mL to about 700 ng·h/mL in the mammal to which the dosage form is administered. This amount may be suitable for administration of the oral dosage form about every 3 to 4 weeks.

In some embodiments, the zoledronic acid, such as zoledronic acid in an acid form or a salt form, such as the disodium salt form, is present in an amount such that the oral dosage form provides an area under the plasma concentration curve (AUC) of zoledronic acid of about 20 ng·h/mL to about 700 ng·h/mL, about 50 ng·h/mL to about 500 ng·h/mL, about 50 ng·h/mL to about 400 ng·h/mL, about 50 ng·h/mL to about 300 ng·h/mL, about 50 ng·h/mL to about 200 ng·h/mL, about 50 ng·h/mL to about 100 ng·h/mL, about 130 ng·h/mL to about 150 ng·h/mL, about 130 ng·h/mL to about 140 ng·h/mL, about 150 ng·h/mL to about 200 ng·h/mL, about 200 ng·h/mL to about 300 ng·h/mL, about 250 ng·h/mL to about 300 ng·h/mL, about 300 ng·h/mL to about 400 ng·h/mL, about 400 ng·h/mL to about 500 ng·h/mL, about 350 ng·h/mL to about 400 ng·h/mL, about 450 ng·h/mL to about 500 ng·h/mL, about 130 ng·h/mL to about 160 ng·h/mL, about 405 ng·h/mL to about 450 ng·h/mL, about 100 ng·h/mL to about 500 ng·h/mL, about 100 ng·h/mL to about 400 ng·h/mL, about 100 ng·h/mL to about 300 ng·h/mL, about 100 ng·h/mL to about 200 ng·h/mL, about 125 ng·h/mL to about 500 ng·h/mL, about 125 ng·h/mL to about 400 ng·h/mL, about 125 ng·h/mL to about 300 ng·h/mL, about 125 ng·h/mL to about 200 ng·h/mL, or about 200 ng·h/mL to about 300 ng·h/mL, in the mammal to which the dosage form is administered. This amount may be suitable for weekly administration of the oral dosage, or for administration of 3 to 5 individual dosages during a month. The individual dosages could be given at regular intervals, given during the first week, or at any other schedule that provides 3 to 5 dosages during the month.

In some embodiments, the zoledronic acid is present in an amount such that the oral dosage form provides an area under the plasma concentration curve of zoledronic acid of about 4 ng·h/mL to about 100 ng·h/mL, about 10 ng·h/mL to about 50 ng·h/mL, about 10 ng·h/mL to about 30 ng·h/mL, 20 ng·h/mL to about 700 ng·h/mL, about 50 ng·h/mL to about 500 ng·h/mL, about 50 ng·h/mL to about 400 ng·h/mL, about 50 ng·h/mL to about 300 ng·h/mL, about 50 ng·h/mL to about 200 ng·h/mL, about 100 ng·h/mL to about 500 ng·h/mL, about 100 ng·h/mL to about 400 ng·h/mL, about 100 ng·h/mL to about 300 ng·h/mL, about 100 ng·h/mL to about 200 ng·h/mL, about 125 ng·h/mL to about 500 ng·h/mL, about 125 ng·h/mL to about 400 ng·h/mL, about 125 ng·h/mL to about 300 ng·h/mL, about 125 ng·h/mL to about 200 ng·h/mL, or about 200 ng·h/mL to about 300 ng·h/mL in the mammal to which the dosage form is administered. This amount may be suitable for daily administration of the oral dosage form. In some embodiments, the dosage form may be administered for 2, 3, 4, 5, 6, 7, 8, 9, or 10, 5 to 10, or 6 to 10 consecutive days.

In some embodiments, the zoledronic acid, such as zoledronic acid in an acid form or a salt form, such as the disodium salt form, is present in an amount such that the oral administration of the dosage form in a fasted state results in an area under the plasma concentration curve (AUC) of zoledronic acid of about 50 ng·h/mL to about 500 ng·h/mL, about 50 ng·h/mL to about 100 ng·h/mL, about 100 ng·h/mL to about 200 ng·h/mL, about 130 ng·h/mL to about 180 ng·h/mL, about 130 ng·h/mL to about 150 ng·h/mL, about 130 ng·h/mL to about 140 ng·h/mL, about 140 ng·h/mL to about 150 ng·h/mL, about 150 ng·h/mL to about 200 ng·h/mL, about 200 ng·h/mL to about 300 ng·h/mL, about 250 ng·h/mL to about 300 ng·h/mL, about 300 ng·h/mL to about 400 ng·h/mL, about 300 ng·h/mL to about 350 ng·h/mL, about 400 ng·h/mL to about 500 ng·h/mL, about 350 ng·h/mL to about 400 ng·h/mL, about 450 ng·h/mL to about 500 ng·h/mL, about 130 ng·h/mL to about 160 ng·h/mL, about 405 ng·h/mL to about 450 ng·h/mL, measured over a 24 hour period.

In some embodiments, a bisphosphonate, such as zoledronic acid (e.g. oral zoledronic acid), etc., is administered at an interval of about once, twice, or thrice daily, or every 1, 2, 3, 4, 5, 6, 7, 8, 9, 10, 11, 12, 13, or 14 days; or 15, 16, 17, 18, 19, 20, or 21 days; or 22, 23, 24, 25, 26, 27, 28, 29, 30, or 31 days; or 32, 33, 34, 35, 36, 37, 38, 39, 40, 41, 42, 43, 44, or 45; or 46, 47, 48, 49, 50, 51, 52, 53, 54, 55, 56, 57, 58, 59, or 60 days; or 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days; or 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 days.

An oral dosage form comprising zoledronic acid having a dose of zoledronic acid and a configuration suitable for a particular species of mammal may be configured so that the particular species of mammal has a plasma concentration of zoledronic acid at 12 hours that is about 0.5 ng/mL to about 5 ng/mL, about 1 ng/mL to about 3 ng/mL, about 1 ng/mL to about 2 ng/mL, about 2 ng/mL to about 3 ng/mL, about 3 ng/mL to about 4 ng/mL, about 1.2 ng/mL, about 2.6 ng/mL, about 3.2 ng/mL, or any plasma concentration in a range bounded by, or between, any of these values.

An oral dosage form comprising zoledronic acid having a dose of zoledronic acid and a configuration suitable for a particular species of mammal may be configured so that the particular species of mammal has a plasma concentration of zoledronic acid at 24 hours that is about 0.2 ng/mL to about 2 ng/mL, about 0.5 ng/mL to about 1.5 ng/mL, about 0.5 ng/mL to about 1 ng/mL, about 1 ng/mL to about 1.5 ng/mL, about 0.5 ng/mL, about 1.0 ng/mL, about 1.4 ng/mL, or any plasma concentration in a range bounded by, or between, any of these values.

An oral dosage form comprising zoledronic acid having a dose of zoledronic acid and a configuration suitable for a particular species of mammal may be configured so that the particular species of mammal has a plasma concentration of zoledronic acid at 36 hours that is about 0.1 ng/mL to about 2 ng/mL, about 0.2 ng/mL to about 1.5 ng/mL, about 0.2 ng/mL to about 0.5 ng/mL, about 0.5 ng/mL to about 1 ng/mL, about 1 ng/mL to about 1.3 ng/mL, about 0.3 ng/mL, about 0.8 ng/mL, about 1.1 ng/mL, or any plasma concentration in a range bounded by, or between, any of these values.

An oral dosage form comprising zoledronic acid having a dose of zoledronic acid and a configuration suitable for a particular species of mammal may be configured so that the particular species of mammal has a plasma concentration of zoledronic acid at 48 hours that is about 0.1 ng/mL to about 2 ng/mL, about 0.2 ng/mL to about 1.5 ng/mL, about 0.2 ng/mL to about 0.5 ng/mL, about 0.5 ng/mL to about 0.9 ng/mL, about 0.9 ng/mL to about 1.3 ng/mL, about 0.3 ng/mL, about 0.7 ng/mL, about 1.1 ng/mL, or any plasma concentration in a range bounded by, or between, any of these values.

An oral dosage form comprising zoledronic acid having a dose of zoledronic acid and a configuration suitable for a particular species of mammal may be configured so that the particular species of mammal has a plasma concentration of zoledronic acid at 72 hours that is about 0.2 ng/mL to about 1 ng/mL, about 0.2 ng/mL to about 1.5 ng/mL, about 0.1 ng/mL to about 0.3 ng/mL, about 0.3 ng/mL to about 0.6 ng/mL, about 0.6 ng/mL to about 1 ng/mL, about 0.2 ng/mL, about 0.5 ng/mL, about 0.9 ng/mL, or any plasma concentration in a range bounded by, or between, any of these values.

An oral dosage form comprising zoledronic acid having a dose of zoledronic acid and a configuration suitable for a particular species of mammal may be configured so that the elimination half-life of zoledronic acid in the particular species of mammal is about 30 hours to about 100 hours, about 40 hours to about 60 hours, about 40 hours to about 50 hours, about 50 hours to about 60 hours, about 42 hours, about 51 hours, about 59 hours, or any half-life in a range bounded by, or between, any of these values.

As used herein, the “elimination half-life” refers to the apparent first-order terminal plasma elimination half-life, obtained by non-compartmental analysis using Win-Nonlin. A terminal plasma elimination half-life is the time required to reduce the plasma concentration to half after reaching pseudo-equilibrium, and not the time required to eliminate half the administered dose. For orally administered drugs, terminal plasma elimination half-life can be affected by absorption of the drug, as well as plasma clearance and extent of distribution.

The C-terminal telopeptide (CTX) is one of the products from type I collagen degradation by osteoclasts during bone resorption. Thus, CTX serum levels may be used as a biomarker to indicate and monitor bone breakdown, resorption, and loss. In some embodiments, zoledronic acid and other bisphosphonates may be used to inhibit osteoclast activity and/or lower CTX serum levels, for example, by at least about 5%, at least about 10%, at least about 15%, at least about 20%, at least about 25%, at least about 30%, at least about 35%, at least about 40%, at least about 45%, at least about 50%, at least about 55%, at least about 60%, at least about 70%, at least about 75%, at least about 80%, at least about 85%, at least about 90%, at least about 95%, at least about 99%, at least about 100%, about 60%-70%, about 70%-80%, about 80%-90%, about 85-95%, about 80%-85%, about 85%-90%, about 90%-95%, or any other reduction in osteoclast activity or CTX serum levels in a range bounded by, or between, any of these values.

Zoledronic acid or another bisphosphonate may be combined with a pharmaceutical carrier selected on the basis of the chosen route of administration and standard pharmaceutical practice as described, for example, in Remington's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

Zoledronic acid or another bisphosphonate may be administered by any means that may result in the contact of the active agent(s) with the desired site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

The effective amount of zoledronic acid or another bisphosphonate will vary depending on various factors known to the treating physicians, such as the severity of the condition to be treated, route of administration, formulation and dosage forms, physical characteristics of the bisphosphonate compound used, and age, weight and response of the individual patients.

The dose of zoledronate or another bisphosphonate compound may be administered in a single or divided doses.

Any suitable amount of bisphosphonate, e.g. zoledronic acid, may be used in a dosage form. For example, some liquid compositions may comprise about 0.0001% (w/v) to about 50% (w/v), about 0.01% (w/v) to about 20% (w/v), about 0.01% to about 10% (w/v), about 0.001% (w/v) to about 1% (w/v), about 0.1% (w/v) to about 0.5% (w/v), about 1% (w/v) to about 3% (w/v), about 3% (w/v) to about 5% (w/v), about 5% (w/v) to about 7% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of zoledronic acid.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 75% (w/w), about 70% (w/w) to about 80% (w/w), or about 80% (w/w) to about 90% (w/w) of zoledronic acid.

Any suitable amount of a bisphosphonate may be used. Some solid or liquid oral dosage forms, or units of oral dosage forms (referred to collectively herein as “oral dosage form(s)”) may contain a molar equivalent to about 1-200 mg, about 1-5 mg, about 2-6 mg, about 3-7 mg, about 4-8 mg, about 5-9 mg, about 6-10 mg, about 7-12 mg, about 8-15 mg, about 10-20 mg, about 15-25 mg, about 20-40 mg, about 30-50 mg, about 40-60 mg, about 45-55 mg, about 48-52 mg, about 50 mg, about 50-70 mg, about 50-100 mg, about 70-120 mg, about 100-150 mg, about 120-170 mg, about 150-200 mg of the bisphosphonate, in an acid form or in a salt form, such as disodium salt form, or any amount of the bisphosphonate in a range bounded by, or between, any of these values. In some embodiments, the oral bisphosphonate is administered daily, weekly, biweekly, monthly, every two or three months, once a year, or twice a year. In some embodiments, the oral or IV bisphosphonate is administered daily, every other day, every third day, weekly, biweekly, monthly, every two or three months, every six months, once a year, or twice a year from day 1.

Some oral dosage forms may contain about 1-200 mg, about 1-5 mg, about 2-6 mg, about 3-7 mg, about 4-8 mg, about 5-9 mg, about 6-10 mg, about 7-12 mg, about 8-15 mg, about 10-20 mg, about 15-25 mg, about 20-40 mg, about 30-50 mg, about 40-60 mg, about 45-55 mg, about 48-52 mg, about 50 mg, about 50-70 mg, about 50-100 mg, about 70-120 mg, about 100-150 mg, about 120-170 mg, about 150-200 mg of zoledronic acid, or any amount of zoledronic acid in a range bounded by, or between, any of these values. Amounts of zoledronic acid (and other bisphosphonates) used anywhere herein are based upon the weight of the diacid form, but also apply to other forms using appropriate adjustments for differences in molecular weight. Thus, for other forms of zoledronic acid, such as a salt form (e.g. the disodium salt form), the amount would be the amount in mg of the other form that would be molar equivalent to the acid form. For example, about 1.2-232 mg of the disodium salt form of zoledronic acid (MW: 316.09 g/mol) is the molar equivalent of about 1-200 mg of the diacid form of zoledronic acid (MW: 272.09 g/mol). In some embodiments, the oral zoledronic acid is administered daily, weekly, biweekly, monthly, every two or three months, once a year, or twice a year. In some embodiments, the oral or IV zoledronic acid is administered daily, every other day, every third day, weekly, biweekly, monthly, every two or three months, every six months, once a year, or twice a year from day 1.

In some embodiments, the daily oral dose of a bisphosphonate, such as a nitrogen-containing bisphosphonate, is about 1-50 mg, about 1-10 mg, about 3-13 mg, about 5-15 mg, about 7-17 mg, about 10-20 mg, about 10-30 mg, about 20-40 mg, about 30-50 mg, or any amount in a range bounded by, or between, any of these values.

In some embodiments, the daily oral dose zoledronic acid, is about 1-20 mg, about 1-10 mg, about 2-12 mg, about 4-14 mg, about 5-10 mg, about 5-20 mg, about 6-8 mg, about 7-8 mg, about 6-16 mg, about 7-17 mg, about 8-18 mg, about 9-19 mg, about 10-20 mg, or any amount of zoledronic acid in a range bounded by any of these values.

In some embodiments, the weekly oral dose of the bisphosphonate, such as a nitrogen-containing bisphosphonate, is about 1-500 mg, about 1-50 mg, about 10-60 mg, about 20-70 mg, about 30-80 mg, about 40-140 mg, about 50-150 mg, about 60-160 mg, about 70-170 mg, about 80-180 mg, about 90-190 mg, about 100-200 mg, about 150-250 mg, about 200-300 mg, about 250-350 mg, about 300-400 mg, about 350-450 mg, about 400-500 mg, or any amount in a range bounded by any of these values. The weekly oral dose may be given as a single dose, given once during the week, or may be given in 2, 3, 4, 5, 6, or 7 individual doses during the week.

In some embodiments the weekly oral dose of zoledronic acid is about 20-100 mg, about 20-40 mg, about 30-50 mg, about 40-60 mg, about 45-55 mg, about 48-52 mg, about 50 mg, about 50-70 mg, about 60-100 mg, about 70-100 mg, or any amount of zoledronic acid in a range bounded by any of these values. The weekly oral dose may be given as a single dose, given once during the week, or may be given in 2, 3, 4, 5, 6, or 7 individual doses during the week.

In some embodiments, the monthly dose of the bisphosphonate, such as a nitrogen-containing bisphosphonate, or the amount of the bisphosphonate that is administered over a period of a month, is about 1-500 mg, about 1-50 mg, about 10-60 mg, about 20-70 mg, about 30-80 mg, about 40-140 mg, about 50-150 mg, about 60-160 mg, about 70-170 mg, about 80-180 mg, about 90-190 mg, about 100-200 mg, about 150-250 mg, about 200-300 mg, about 250-350 mg, about 300-400 mg, about 350-450 mg, about 400-500 mg, about 450-550 mg, about 500-600 mg, about 550-650 mg, about 600-800 mg, about 700-900 mg, about 800-1000 mg, or any monthly dose in a range bounded by any of these values. A monthly dose may be given as a single dose, or as two or more individual doses administered during the month. In some embodiments, the monthly dose is administered in 2 or 3 weekly doses. In some embodiments, the monthly dose is administered in 4 or 5 weekly doses. In some embodiments, the monthly dose is administered in 28 to 31 daily doses. In some embodiments, the monthly dose is administered in 5 to 10 individual doses during the month. The monthly dose may be administered for only 1 month, or may be repeatedly administered for 2 or more months.

In some embodiments, the monthly dose of zoledronic acid, or the amount of zoledronic acid that is administered over a period of a month, or a period of 4 weeks, is about 50-150 mg, about 100-200 mg, about 150-250 mg, about 160-240 mg (particularly for a period of 4 weeks), about 180-220 mg (particularly for a period of 4 weeks), about 200 mg (particularly for a period of 4 weeks), about 100-300 mg, about 150-300 mg, about 200-300 mg, about 250-350 mg, about 300-500 mg, about 400-600 mg, about 500-700 mg, about 600-1000 mg, or any amount of zoledronic acid in a range bounded by any of these values. A monthly dose may be given as a single dose, or as two or more individual doses administered during the month. In some embodiments, the monthly dose is administered in 2 or 3 weekly doses. In some embodiments, the monthly dose is administered in 4 or 5 weekly doses. In some embodiments, the monthly dose is administered in 28 to 31 daily doses. In some embodiments, the monthly dose is administered in 5 to 10 individual doses during the month. The monthly dose may be administered for only 1 month, or may be repeatedly administered for 2 or more months.

In some embodiments, a six week dose of zoledronic acid may be about 200-500 mg, about 300-450 mg, about 280-320 mg, or about 300 mg. In some embodiments, the six week dose of zoledronic acid may be administered only once. In some embodiments, the six week dose of zoledronic acid may be administered in six weekly doses, e.g about 35 mg to about 80 mg or about 50 mg to about 75 mg in each weekly dose.

In some embodiments, about 10 mg to about 150 mg of zoledronic acid in an acid form or a salt form may be orally administered to a mammal, such as a human being, once weekly for about 6 weeks. In some embodiments, about 40 mg to about 60 mg of zoledronic acid in an acid form or a salt form may be orally administered to a mammal, such as a human being, once weekly for about 6 weeks.

With respect to oral administration of zoledronic acid or another bisphosphonate, it may be helpful to administer the drug in a manner that maximizes oral bioavailability. For example, orally administering zoledronic acid or another bisphosphonate in a fasted state can potentially help to maximize bioavailability. In some embodiments, the person taking the drug orally, is fasted, or does not eat or drink (other than any water required to swallow the oral dosage form), for at least about 1 hour, at least about 2 hours, at least about 4 hours, at least about 6 hours, at least about 8 hours, at least about 10 hours, or at least about 12 hours before the bisphosphonate is administered. It may also be helpful if the mammal or human being to which the zoledronic acid or other bisphosphonate is administered does not eat or drink for at least about 30 minutes, at least about 1 hour, at least about 2 hours, at least about 3 hours, or at least about 4 hours after the bisphosphonate is administered. In some embodiments, a human being to which the zoledronic acid is administered avoids lying down, or remains upright or sits upright, for at least about 30 minutes or about 1 hour after receiving a dosage form containing the bisphosphonate.

A bisphosphonate, such as zoledronic acid, may be formulated for oral administration, for example, with an inert diluent or with an edible carrier, or it may be enclosed in hard or soft shell gelatin capsules, compressed into tablets, or incorporated directly with the food of the diet. For oral therapeutic administration, the active compound may be incorporated with an excipient and used in the form of ingestible tablets, buccal tablets, coated tablets, troches, capsules, elixirs, dispersions, suspensions, solutions, syrups, wafers, patches, and the like.

Tablets, troches, pills, capsules and the like may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the unit dosage form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for material in a dosage form or pharmaceutical composition to be pharmaceutically pure and substantially non-toxic in the amounts employed.

A bisphosphonate, such as zoledronic acid, may be formulated for parental or intraperitoneal administration. Solutions of the active compounds as free acids or pharmacologically acceptable salts can be prepared in water suitably mixed with a surfactant, such as hydroxypropylcellulose. A dispersion can also have an oil dispersed within, or dispersed in, glycerol, liquid polyethylene glycols, and mixtures thereof. Under ordinary conditions of storage and use, these preparations may contain a preservative to prevent the growth of microorganisms.

An example of a useful composition for a dosage form containing about 20-200 mg of zoledronic acid is shown in Table A below:

TABLE A

| Component | Amount (wt/wt) |
|---|---|
| zoledronic acid | 30-70% |
| lubricant | 1-10% |
| diluent | 20-70% |
| disintegrant | 1-10% |

In some embodiments, an oral dosage form may comprise a silicified microcrystalline cellulose such as PROSLOV®. For example, about 20% (wt/wt) to about 70% (wt/wt), about 10% (wt/wt) to about 20% (wt/wt), about 20% (wt/wt) to about 40% (wt/wt), about 25% (wt/wt) to about 30% (wt/wt), about 40% (wt/wt) to about 50% (wt/wt), or about 45% (wt/wt) to about 50% (wt/wt) silicified microcrystalline cellulose may be present in an oral dosage form or a unit of an oral dosage form.

In some embodiments, an oral dosage form may comprise a crosslinked polyvinylpyrrolidone such as crospovidone. For example, about 1% (wt/wt) to about 10% (wt/wt), about 1% (wt/wt) to about 5% (wt/wt), or about 1% (wt/wt) to about 3% (wt/wt) crosslinked polyvinylpyrrolidone may be present in an oral dosage form or a unit of an oral dosage form.

In some embodiments, an oral dosage form may comprise a fumed silica such as AEROSIL®. For example, about 0.1% (wt/wt) to about 10% (wt/wt), about 0.1% (wt/wt) to about 1% (wt/wt), or about 0.4% (wt/wt) to about 0.6% (wt/wt) fumed silica may be present in an oral dosage form or a unit of an oral dosage form.

In some embodiments, an oral dosage form may comprise magnesium stearate. For example, about 0.1% (wt/wt) to about 10% (wt/wt), about 0.1% (wt/wt) to about 1% (wt/wt), or about 0.4% (wt/wt) to about 0.6% (wt/wt) magnesium stearate may be present in an oral dosage form or a unit of an oral dosage form.

An oral dosage form comprising zoledronic acid or another bisphosphonate may be included in a pharmaceutical product comprising more than one unit of the oral dosage form.

A pharmaceutical product containing oral dosage forms for daily use can contain 28, 29, 30, or 31 units of the oral dosage form for a monthly supply. An approximately 6 week daily supply can contain 40 to 45 units of the oral dosage form. An approximately 3 month daily supply can contain 85 to 95 units of the oral dosage form. An approximately six month daily supply can contain 170 to 200 units of the oral dosage form. An approximately one year daily supply can contain 350 to 380 units of the oral dosage form.

A pharmaceutical product containing oral dosage forms for weekly use can contain 4 or 5 units of the oral dosage form for a monthly supply. An approximately two month weekly supply can contain 8 or 9 units of the oral dosage form. An approximately six week weekly supply can contain about 6 units of the oral dosage form. An approximately three month weekly supply can contain 12, 13 or 14 units of the oral dosage form. An approximately six month weekly supply can contain 22 to 30 units of the oral dosage form. An approximately one year weekly supply can contain 45 to 60 units of the oral dosage form.

A pharmaceutical product may accommodate other dosing regimens. For example, a pharmaceutical product may comprise 5 to 10 units of the oral dosage form, wherein each unit of the oral dosage form contains about 40 mg to about 150 mg of zoledronic acid. Some pharmaceutical products may comprise 1 to 10 units of the oral dosage form, wherein the product contains about 200 mg to about 2000 mg of zoledronic acid. For such a product, each unit of the oral dosage form may be taken daily for 1 to 10 days or 5 to 10 days during a month, such as at the beginning of a month.

Some oral dosage forms comprising a suitable bisphosphonate like zoledronic acid, or salts thereof—may have enteric coatings or film coatings. In some embodiments, an oral dosage form of a bisphosphonate comprises a tablet having an enteric coating. In some embodiments, an oral dosage form of a bisphosphonate comprises a capsule having an enteric coating. In some embodiments, an oral dosage form of a bisphosphonate comprises a tablet having a film coating. In some embodiments, an oral dosage form of a bisphosphonate comprises a capsule having a film coating.

### Example 1. Bioavailability of Orally Administered Zoledronic Acid and Disodium Zoledronate

Tablets were manufactured containing zoledronic acid in either the diacid form or the disodium salt form (disodium zoledronate tetrahydrate). Both types of tablets contained 50 mg of zoledronic acid equivalent per tablet. Identical excipients were used in both types of tablets, with amounts adjusted to account for the difference in molecular weights between the acid and the disodium salt.

Beagle dogs were orally administered tablets containing 150 mg zoledronic acid equivalent either in the disodium salt form of zoledronate (Group 1) or the diacid form of zoledronic acid (Group 2). Each animal was given three 50 mg equivalent tablets (150 mg total), which were administered together. The animal's oral cavity was wetted with water before placing the tablets on the back of the animal's tongue. Animals were fasted before and after dosing. Animals were 6 to 9 months of age and weighed 6 to 10 kg on the day of dosing. There were three dogs per group.

Serial blood samples were collected from each animal by venipuncture of the jugular vein at various points after dosing for measurement of plasma concentrations of zoledronic acid. Blood samples were collected into chilled tubes containing K₂EDTA as the anticoagulant. Samples were then centrifuged at approximately 3000 rpm at +4° C. for 10 minutes for plasma derivation. Plasma concentrations of zoledronic acid were measured using an LC/MS/MS method.

### Results

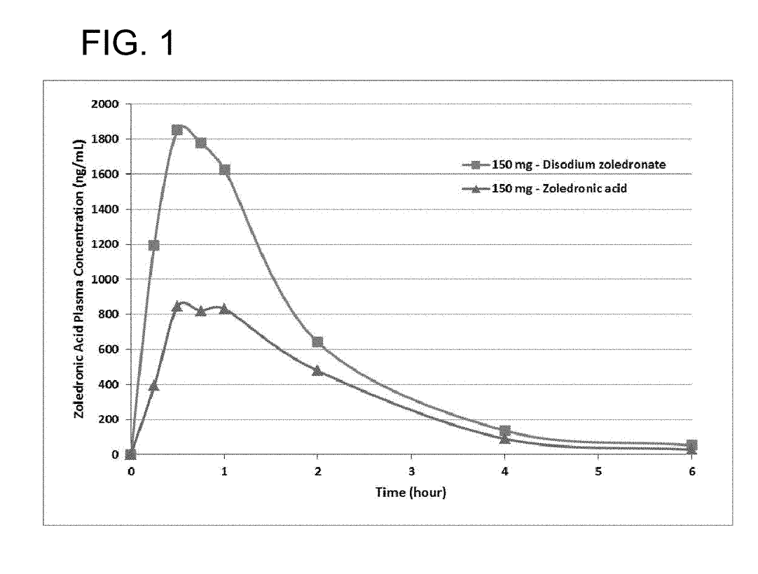

The average plasma concentrations of zoledronic acid for each group of dogs is summarized in Table 1 and illustrated in FIG. 1. Detectable plasma levels of zoledronic acid were observed for the entire 48 hours that they were measured.

TABLE 1
Zoledronic Acid plasma concentrations in beagle dogs

| | | | Plasma |
|---|---|---|---|
| | | Time | concentration |
| | | (hour) | (ng/mL) |
| Group 1 (N = 3) | Disodium Zoledronate | 0 | 0.00 |
| | Tablets | 0.25 | 1193.97 |
| | (150 mg acid equivalent) | 0.5 | 1852.12 |
| | | 0.75 | 1776.51 |
| | | 1 | 1626.56 |
| | | 2 | 640.57 |
| | | 4 | 136.93 |
| | | 6 | 53.11 |
| | | 8 | 26.97 |
| | | 12 | 13.74 |
| | | 24 | 6.78 |
| | | 48 | 5.39 |
| Group 2 (N = 3) | Zoledronic Acid Tablets | 0 | 0.00 |
| | (150 mg acid equivalent) | 0.25 | 390.92 |
| | | 0.5 | 846.19 |
| | | 0.75 | 819.15 |
| | | 1 | 831.77 |
| | | 2 | 477.76 |
| | | 4 | 90.11 |
| | | 6 | 28.22 |
| | | 8 | 15.10 |
| | | 12 | 6.13 |
| | | 24 | 3.18 |
| | | 48 | 1.84 |

The disodium salt form of zoledronic acid produced significantly higher plasma levels of zoledronic acid than the diacid form of zoledronic acid, indicating improved oral absorption with the disodium salt form. Measured using peak plasma concentrations (Cₘₐₓ), the disodium salt resulted in a 119% actual and 74% weight-adjusted increase in bioavailability as compared to the diacid form of zoledronic acid. Measured using area under the plasma concentration curve (AUC_{0-∞}), bioavailability was 84% and 46% greater with the disodium salt than with pure zoledronic acid, on an actual and weight-adjusted basis respectively. The average AUC_{0-∞} for the disodium salt was 4073 ng·h/mL and the average AUC_{0-∞} for the diacid was 2217 ng·h/mL. The AUC_{0-∞} was found to be dose proportional. Thus, for beagle dogs similar to those tested, about 3 mg to about 4 mg of the disodium salt would be expected to result in an AUC_{0-∞} of about 100 ng·h/mL, and about 7 mg to about 8 mg of the disodium salt would be expected to result in an AUC_{0-∞} of about 200 ng·h/mL.

### Example 2

### Methods

A study was performed to evaluate the efficacy of a single intravenous infusion of 5 mg zoledronic acid in comparison with intravenous placebo infusion among patients with chronic low back pain (LBP) and Modic changes on MRI. This study was a double-blinded, randomized, placebo-controlled clinical trial in patients with low back pain (LBP). Patients were included in the study if they had low back symptoms for at least three months, a LBP of at least six (6) on a 10-cm Visual Analog Scale (VAS) or an Oswestry Disability Index (ODI) of at least 30%, and an M1, mixed M1/2 or M2 type change on MRI performed within six months at most prior to enrollment.

Patients were excluded from the study if they had renal impairment with reduced creatinine clearance defined as an estimated glomerular filtration rate (eGFR) below 40 ml/min, hypocalcemia, known hypersensitivity to zoledronic acid or other bisphosphonates or ingredients of the infusion product, the presence of red flags, nerve root entrapment or willingness for early retirement. Premenopausal women of childbearing potential were also excluded. Blood samples were taken prior to the infusion to assess the serum concentration of calcium and creatinine. The clinical examination included medical history and clinical assessment of lumbar flexibility, tendon signs, and motor and sensory testing.

After confirmation of eligibility patients were randomized to receive a single intravenous infusion of 5 mg zoledronic acid (n=20) or 100 ml saline as placebo (n=20) over a 15-minute period. Information on use of the concomitant medication and hospital admissions were recorded. Blood samples were taken for the assessment of safety, inflammatory mediators and markers of bone turnover at baseline, at one month and at one year.

Clinical assessments were performed at 14 days before enrollment (screening visit), and at follow-up visits at one month and one year after the infusion. The primary outcome was the change in the intensity of LBP on VAS. Secondary outcomes included leg pain intensity, ODI, health-related quality of life assessed with RAND-36, patient-reported sick leaves and lumbar flexibility. These outcome measures were assessed at baseline and at each follow-up. Lumbar flexibility was evaluated using the fingers-to-floor and trunk side bending measures (in cm). The percentage of patients undergoing a 20% relative improvement, the proportion of patients reaching a VAS score of 40 mm or less using 100 mm scale in the primary outcome, and patient acceptable symptom state (PASS) were also assessed. Pain medication use was inquired about during the follow-up visits.

### Results

Zoledronic acid treatment resulted in a greater improvement in LBP intensity at one month as compared to placebo treatment. Furthermore, the patients receiving zoledronic acid reported NSAID use at one year significantly less often than those in the placebo group. Overall, the improvements in most of the evaluated parameters were greater in the zoledronic acid group throughout the follow-up period.

The clinical characteristics of study participants at baseline are displayed in Table 2. The mean LBP duration was 293 days, initial LBP intensity on VAS 6.7, leg pain on VAS 2.9 and the ODI score was 32%. Altogether 19 patients in the ZA group and 18 in the placebo group had a M1/2 lesion. Modic changes were most commonly (70%) situated at L4/5 or L5/S1. The zoledronic acid and placebo groups were similar as regards the demographic and background characteristics of all patients at baseline (Table 2).

The mean difference (MD) between the treatment groups in the primary outcome, intensity of LBP, significantly favored zoledronic acid at one month (MD 1.4; 95% Cl 0.01 to 2.9) while at one year no significant difference was observed (MD 0.7; 95% CI −1.0 to 2.4; Table 3). The proportion of patients with at least 20% improvement in intensity of LBP and PASS both favored the zoledronic acid treatment at one month: zoledronic acid 55% vs. placebo 25% (p=0.105) and zoledronic acid 50% vs. placebo 20% (p=0.096), respectively.

For the patients who were treated with zoledronic acid, the reduction in pain intensity was greater in those with greater baseline pain intensity as shown in Table 5. The mean reduction in pain from baseline was 3.4 for patients with baseline pain intensity ≥7, as compared to a reduction of only 0.1 for patients with a baseline pain intensity <6.

Of the secondary outcomes, the improvement in ODI, favored zoledronic acid at 1 month, the adjusted between-group difference being 6.0% (95% CI −0.6 to 13), but not at one year (Table 3). Similarly, side bending (to right and left) favored the zoledronic acid treatment at one month but not at one year (Table 3). Changes in total RAND-36, and in the physical and mental components of RAND-36 are shown in Table 4.

At baseline, there were no differences in self-reported use of non-steroidal anti-inflammatory drugs (NSAIDs) between the treatment groups, whereas at one year, only 20% of patients in the ZA group used NSAIDs versus 60% in the placebo group.

TABLE 2
Baseline characteristics of study population
according to treatment group

| | Zoledronic Acid | | Pacebo | |
|---|---|---|---|---|
| Characteristics | n = 20 | | n = 20 | |
| Sex, n (%) men | 15 | (75) | 11 | (55) |
| Age, mean (SD) years | 49 | (9.3) | 51 | (7.3) |
| Smoking, n (%) regular smokers* | 5 | (25) | 6 | (30) |
| BMI, mean (SD) kg/m | 26 | (3.3) | 27 | (3.2) |
| Workload, n (%) | | | | |
| Sedentary work with limited | 4 | (20) | 4 | (22) |
| walking | | | | |
| Fairly light work with considerable | 4 | (20) | 3 | (17) |
| walking but no lifting or carrying | | | | |
| heavy objects | | | | |
| Fairly strenuous work with walking | 8 | (40) | 6 | (33) |
| and lifting heaving objects or | | | | |
| climbing stairs or uphill | | | | |
| Very strenuous work with lifting or | 4 | (20) | 5 | (28) |
| carrying heaving objects such as | | | | |
| shoveling, digging, or hammering | | | | |
| Type of worst MC-lesion**, n | | | | |
| Type I | 1 | | 1 | |
| Type I/II | 19 | | 18 | |
| Type II | 0 | | 1 | |
| MC at two or more levels, n (%) | 7 | (3.5) | 4 | (20) |
| Levels of MC, n | | | | |
| L2/3 | 4 | | 0 | |
| L3/4 | 3 | | 5 | |
| L4/5 | 6 | | 5 | |
| L5/S1 | 7 | | 10 | |
| Duration of LBP, median (IQ | 330 | (200, 365) | 315 | (270, 365) |
| range) days | | | | |
| Intensity of LBP, mean (SD)*** | 6.6 | (1.4) | 6.8 | (1.6) |
| Duration of leg pain, median (IQ | 50 | (0, 100) | 36 | (0, 160) |
| range) days | | | | |
| Intensity of leg pain, mean | 3.0 | (3.1) | 2.9 | (2.3) |
| (SD)*** | | | | |
| Oswestry Disability Index, %, | 30 | (11) | 35 | (10) |
| Mean (SD) | | | | |
| Duration of sick leave during the | 14 | (0, 48) | 18 | (1, 181) |
| past year, median (IQ range) days | | | | |
| RAND-36, mean (SD) | 50 | (8) | 50 | (7) |
| RAND-36 physical component, | 51 | (8) | 49 | (8) |
| mean (SD) | | | | |
| RAND-36 mental component, | 51 | (8) | 49 | (9) |
| mean (SD) | | | | |

BMI = Body Mass Index, MC = Modic Change, LBP = low back pain, SD = standard deviation, IQ = inter-quartile.
*Smoking at least one cigarette per day.
**If different types of MC at two or more levels, classification is based on the assumed severity of the type, i.e., Type I > mixed Type I/II > Type II.
***Assessed using a 10 cm Visual Analogue Scale (VAS).

TABLE 3
Low back symptoms and lumbar flexibility at baseline, one month and 12 months according
to treatment group and between group comparisons of difference from baseline to one month and
12 months

| Mean (SD) original | | | | Unadjusted | | | |
|---|---|---|---|---|---|---|---|
| values | | | | analyses | | Adjusted analyses | |
| ZA | Placebo | Mean (SD) change | | Difference | | Difference | |
| n = 20 | n = 20 | ZA | Placebo | (95% CI) | P | (95% CI) | P* |

Intensity of LBP

| | | | | | | | | |
|---|---|---|---|---|---|---|---|---|
| Baseline | 6.6 (1.4) | 6.8 (1.6) | | | | | | |
| 1 mo. | 4.3 (2.3) | 5.8 (2.2) | −2.2 (2.7) | −0.9 (2.1) | 1.3 | 0.097 | 1.4 | 0.049 |
| | | | | | (−0.2 to | | (0.01 to | |
| | | | | | 2.8) | | 2.9) | |
| 12 mos. | 3.8 (2.5) | 4.6 (2.9) | −2.8 (2.9) | −2.2 (2.5) | 0.6 | 0.474 | 0.7 | 0.387 |
| | | | | | (−1.1 to | | (−1.0 to | |
| | | | | | 2.4) | | 2.4) | |

Intensity of leg pain^{a}

| | | | | | | | | |
|---|---|---|---|---|---|---|---|---|
| Baseline | 3.0 (3.1) | 2.9 (2.3) | | | | | | |
| 1 mo. | 2.0 (2.3) | 3.0 (2.4) | −0.6 (2.4) | 0.1 (2.6) | 0.8 | 0.367 | 0.8 | 0.237 |
| | | | | | (−0.9 to | | (−0.6 to | |
| | | | | | 2.4) | | 2.2) | |
| 12 mos. | 2.1 (2.8) | 2.7 (2.6) | −0.9 (3.4) | −0.3 (3.0) | 0.6 | 0.573 | 0.5 | 0.573 |
| | | | | | (−1.5 to | | (−1.3 to | |
| | | | | | 2.7) | | 2.2) | |

Oswestry disability index, %

| | | | | | | | | |
|---|---|---|---|---|---|---|---|---|
| Baseline | 30 (11) | 35 (10) | | | | | | |
| 1 mo. | 24 (10) | 33 (13) | −5.9 (11) | −1.7 (9.7) | 4.3 | 0.212 | 6.0 | 0.071 |
| | | | | | (−2.5 to 11) | | (−0.6 to | |
| | | | | | | | 13) | |
| 12 mos. | 25 (13) | 33 (15) | −5.0 (15) | −1.9 (12) | 3.1 | 0.475 | 5.1 | 0.231 |
| | | | | | (−5.6 to 12) | | (−3.4 to | |
| | | | | | | | 14) | |

Fingers-to-floor, cm

| | | | | | | | | |
|---|---|---|---|---|---|---|---|---|
| Baseline | 23 (19) | 19 (18) | | | | | | |
| 1 mo. | 17 (17) | 19 (17) | −5.1 (20) | −0.1 (8.3) | 5.0 | 0.306 | 3.6 | 0.403 |
| | | | | | (−4.8 to 15) | | (−5.0 to | |
| | | | | | | | 12) | |
| 12 mos. | 16 (16) | 20 (19) | −6.3 (23) | 0.9 (11) | 7.1 | 0.215 | 5.3 | 0.277 |
| | | | | | (−4.3 to 18) | | (−4.5 to | |
| | | | | | | | 15) | |

Sidebending to right, cm

| | | | | | | | | |
|---|---|---|---|---|---|---|---|---|
| Baseline | 14.1 (4.9) | 13.8 (7.2) | | | | | | |
| 1 mo. | 15.7 (5.9) | 13.3 (6.9) | 1.5 (4.7) | −0.5 (2.2) | −2.0 | 0.101 | −2.0 | 0.087 |
| | | | | | (−4.3 to | | (−4.4 to | |
| | | | | | 0.4) | | 0.3) | |
| 12 mos. | 15.7 (5.6) | 13.8 (6.5) | 1.6 (4.8) | −0.1 (3.5) | −1.6 | 0.227 | −1.7 | 0.180 |
| | | | | | (−4.3 to | | (−4.2 to | |
| | | | | | 1.1) | | 0.8) | |

Sidebending to left, cm

| | | | | | | | | |
|---|---|---|---|---|---|---|---|---|
| Baseline | 15.0 (5.4) | 13.3 (5.5) | | | | | | |
| 1 mo. | 16.1 (5.3) | 12.8 (5.9) | 1.1 (3.0) | −0.5 (2.2) | −1.5 | 0.072 | −1.7 | 0.051 |
| | | | | | (−3.2 to | | (−3.4 to | |
| | | | | | 0.1) | | 0.0) | |
| 12 mos. | 16.2 (6.7) | 13.7 (5.7) | 1.2 (5.3) | 0.5 (3.2) | −0.7 | 0.601 | −1.0 | 0.458 |
| | | | | | (−3.5 to | | (−3.8 to | |
| | | | | | 2.1) | | 1.8) | |

SD = standard deviation,
CI = confidence interval,
ZA = zoledronic acid,
LBP = low back pain.
*ANCOVA: Difference between follow-up and baseline, treatment effect adjusted for baseline value.
^{a}One subject missing at baseline in placebo group and in ZA group, and one subject at 1 month in ZA group.

TABLE 4
Health-related quality of life assessed using RAND-36 at baseline, one month, and 12
months according to treatment group and between group comparisons of difference
from baseline to one month and 12 months

| Mean | | | | | | | |
|---|---|---|---|---|---|---|---|
| (SD) original | | | | Unadjusted | | | |
| values | | Mean (SD) | | analyses | | Adjusted analyses | |
| ZA | Placebo | change | | Difference | | Difference | |
| n = 20 | n = 20 | ZA | Placebo | (95% CI) | P | (95% CI) | P* |

Total RAND-36

| | | | | | | | | |
|---|---|---|---|---|---|---|---|---|
| Baseline | 50 (8) | 50 (7) | | | | | | |
| 1 mo. | 51 (8) | 49 (8) | 0.6 | −0.6 | 1.2 (−3 to 5) | 0.530 | 1.3 (−3 to 5) | 0.477 |
| | | | (6.4) | (5.0) | | | | |
| 12 mos. | 51 (8) | 49 (9) | 1.0 | −1.0 | 2.1 (−3 to 7) | 0.378 | 2.2 (−2 to 7) | 0.314 |
| | | | (8.7) | (5.9) | | | | |

Physical component

| | | | | | | | | |
|---|---|---|---|---|---|---|---|---|
| Baseline | 52 (8) | 48 (8) | | | | | | |
| 1 mo. | 52 (9) | 48 (8) | 0.1 | −0.1 | 0.3 (−4 to 5) | 0.897 | 1.3 (−3 to 6) | 0.554 |
| | | | (8.6) | (5.5) | | | | |
| 12 mos. | 52 (8) | 48 (2) | 0.3 | −0.3 | 0.7 (−5 to 6) | 0.808 | 2.1 (−3 to 7) | 0.405 |
| | | | (10) | (6.5) | | | | |

Mental component

| | | | | | | | | |
|---|---|---|---|---|---|---|---|---|
| Baseline | 49 (9) | 51 (8) | | | | | | |
| 1 mo. | 50 (9) | 50 (9) | 1.0 | −1.0 | 2.0 (−2 to 6) | 0.286 | 1.6 (−2 to 5) | 0.396 |
| | | | (6.1) | (5.6) | | | | |
| 12 mos. | 51 (9) | 49 (9) | 1.8 | −1.8 | 3.5 (−2 to 9) | 0.167 | 2.7 (−2 to 7) | 0.261 |
| | | | (9.0) | (6.7) | | | | |

SD = standard deviation,
CI = confidence interval,
ZA = zoledronic acid.
*ANCOVA: Difference between follow-up and baseline, treatment effect adjusted for baseline value.

TABLE 5
Pain Reduction in Patients Treated Zoledronic Acid (cm)

| | VAS Change from Baseline |
|---|---|
| Baseline VAS <6 | −0.1 |
| Baseline VAS ≥6 and <7 | −2.3 |
| Baseline VAS ≥7 | −3.4 |

### Example 3

This study was designed to compare changes in size and type of MC over one year after a single intravenous infusion of 5 mg zoledronic acid (ZA) vs placebo among chronic LBP patients with MC on MRI, and evaluate whether MRI changes correlate with improvement in clinical symptoms.

### Methods:

### Study Population

The study population consisted of patients with chronic LBP and MC on MRI. Inclusion criteria were LBP for at least three months, LBP intensity of at least six on a 10-cm Visual Analog Scale (VAS) or Oswestry Disability Index (ODI) of at least 30%, and a MC on MRI performed within at most six months prior to enrollment. The exclusion criteria included renal impairment, hypocalcaemia, hypersensitivity to bisphosphonates or the infusion, the presence of red flags, nerve root entrapment, willingness for early retirement, and childbearing potential.

All patients (N=20 in both intervention groups) had MRI at baseline (0.23-1.5 T) and one year (1.5-3 T). We evaluated the level, type, volume (cm³) and proportion of M1 and M2 of all MC. MC were classified to pure M1 (M1 (100%)), mixed M1/2 with predominating M1 (M1/2 (65:35%)) or predominating M2 (M1/2 (35:65%)), and pure M2 (M2 (100%)). The first two were considered M1-dominant, and the latter two M2-dominant. Volumes of M1 and M2 were calculated separately for the primary MC, assumed to cause the symptoms, and for other MC. Cross tabulations were used to describe the distributions of MC type in ZA and placebo groups at baseline and one year. The treatment differences in M1 and M2 volumes, from baseline to one year, were analyzed using ANCOVA with adjustments for age, sex, body mass index and smoking. The correlations of MRI changes with changes in intensity of LBP (10-cm Visual Analog Scale) and Oswestry Disability Index (ODI) were analyzed using Pearson correlations.

### Treatment Intervention

Patients were randomized to receive a single intravenous infusion of 5 mg ZA in 100 ml saline (n=20) or 100 ml saline as placebo (n=20) over a 15-minute period. Before administration of the infusion, all patients received oral ibuprofen 600 mg or paracetamol 1 g to prevent acute phase reactions, and 100 000 units of Vitamin D (Vigantol®) to prevent hypocalcaemia. Patients, the principal investigator, performing the screening and follow-up assessments, and the radiologist evaluating the MRI scans were blinded to the treatment allocation.

### Magnetic Resonance Imaging

Baseline imaging was performed on average 4 months (standard deviation (SD) 3 months, range 0.4 to 11.5 months) before the infusion. Follow-up scans were obtained on average 11.9 months (SD 0.6, range 11 to 13 months) after the infusion with on average 15.9-month (SD 3.2, range 12.1 to 23.5 months) interval between the imagings. Baseline MRIs were obtained with five 1.5 T units and a 0.23 T unit. There was some variation in imaging protocols due to the multiple units used. Protocols were of clinical imaging purpose established to spine imaging. The imaging parameters of sagittal T1-weighted (T1W) turbo spin-echo (TSE) or fast spin-echo (FSE) sequences with fluid attenuation inversion recovery (FLAIR) were repetition time (TR) 1800-2270 ms/inversion time (TI) 860 ms/echo time (TE) 9-29 ms (N=16) and without FLAIR: TR 326-793 ms/TE 8-18 ms (N=23). The imaging parameters of sagittal T2-weighted (T2W) TSE/FSE sequences were TR 3000-4500 ms/TE 105-130 ms (N=40). The imaging parameters of short tau inversion recovery sequences (STIR) were e.g. TR 3400/TI 150/TE 70. Spacing, including slice thickness and slice gap, of the image slices was 4.4-6.2 mm in all sequences.

At the one-year follow-up, MRIs were obtained with two 1.5 T units and a 3 T unit. The imaging parameters of sagittal T1-weighted TSE or FSE sequences with FLAIR were TR 2047-2270 ms/TI 860-900 ms/TE 9-29 ms (N=38) and without FLAIR: TR 540-587/TE 12-24 ms (N=2). The imaging parameters of sagittal T2-weighted TSE or FSE sequences were TR 2796-3500 ms/TE 101-123 ms (N=40). Spacing was 3.6-5 mm in all sequences.

### Image Analysis

MRIs were analyzed for type and volume of each MC from sagittal images. Assessment of type of MC was done using T1W and T2W images, and the MRI scans were classified as previously described: M1 show low signal intensity (SI) on T1W and high SI on T2W and STIR images, M2 show high SI on both T1W and T2W images and low SI on STIR images, and M3 show low SI on both T1W and T2W. T1W images were missing from one patient's image protocol at baseline. Thus the assessment of the type of MC was made from sagittal T2W and STIR images in the case of this patient.

Type of MC was divided in four groups: pure M1 (100%), predominating M1 (M1/2 (65:35%)), predominating M2 (M1/2 (35:65%)) and pure M2 (100%). The first two were considered M1-dominant, and the latter two M2-dominant. Pure M1 and pure M2 were defined to consist almost totally of edemic or fatty changes, respectively, while predominating M1 and predominating M2 were defined as mixed changes with more edemic or fatty signal changes, respectively. The classification was data-driven. The proportion of M3 was so low that it was excluded from the analyses. Area (cm²) of MC was measured slice-by-slice from T2W images by workstation's area tool. The volume (cm³) of MC was calculated by multiplying area with spacing.

Since there were multiple MC in some individuals, a primary MC was defined to represent the most likely LBP generator. Severity of the lesion was assumed as follows: pure M1>predominating M1>predominating M2>pure M2. In case of same types at different levels among patients with multiple MC, the larger MC was selected as the primary MC. The characteristics of the primary MC and other MC were evaluated separately.

Inter-observer reliability was substantial for raw MC type classification (pure M1, predominating M1, predominating M2 and pure M2; linearly weighted kappa 0.62) and also for dichotomized data (M1-dominant vs. M2-dominant; kappa 0.74). Reliability of volume measurements was almost perfect (intra-class correlation coefficient 0.93).

### Statistical Analysis

Baseline characteristics were described using frequencies with proportions, mean values with SD or median values with interquartile range, separately for ZA and placebo groups. Cross tabulations were used to describe the distributions of MC type in ZA and placebo groups at baseline and one year. The differences between the ZA and placebo groups in MC type at baseline and in the change of the MC type during the follow-up were analyzed using the Chi square test. MC volumes at baseline and the change of the MC volumes between the treatment groups were compared using t-test. Additionally, the differences between the ZA and placebo groups in the change of the MC volumes were compared adjusted for age, sex, body mass index and smoking using analysis of covariance. The correlations between change in MRI volumes and changes in intensity of LBP and ODI were analyzed using Pearson correlations.

### Results:

### The Study Population

All 40 enrolled, eligible patients completed the one-year follow-up. The ZA and placebo groups were similar in clinical characteristics at baseline. The patients had a mean age of 50 and a mean BMI of 26.8. The median duration of LBP was 330 days and the mean VAS-score for LBP was 6.7 cm.

### MRI Findings

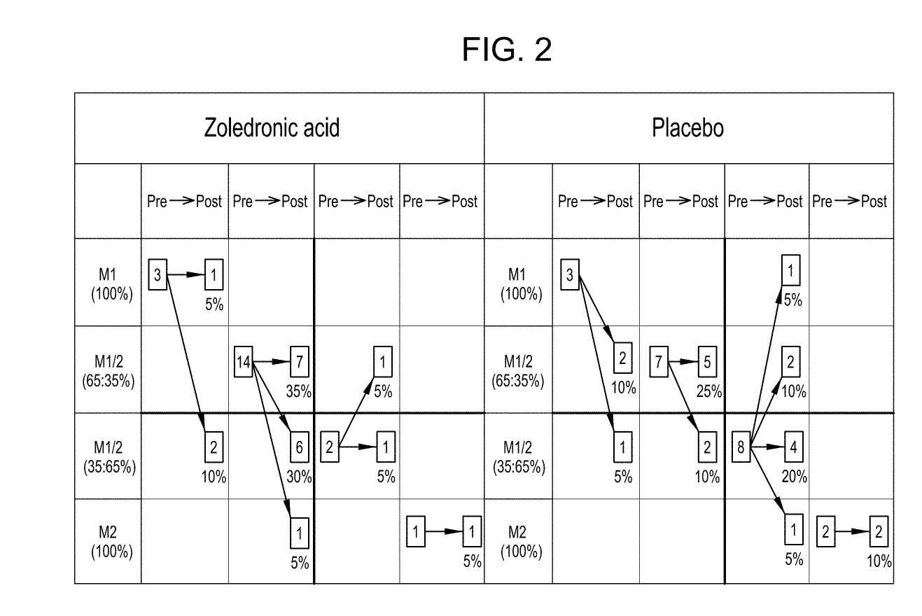

Primary MC occurred most commonly (73%) at L4/5 and L5/S1 (Table 6). At baseline, 6 (15%) patients had pure M1 (100%), 21 (52.5%) had mixed M1/2 with predominating M1 (65:35%), 10 (25%) had mixed M1/2 with predominating M2 (35:65%), and 3 (7.5%) had pure M2 (100%) (FIG. 2). Total volume of MC was 11.4 cm³ at baseline and 13.7 cm³ at 1 year. M1-dominant MC were more common in the ZA group (n=17, 85%) than in the placebo group (n=10, 50%; p=0.041). In the ZA-group 9 (45%) M1-dominant MC converted to M2-dominant and in the placebo group only 3 (15%) (p=0.087; FIG. 2).

The total volume (ZA and placebo groups) of the primary MC at baseline was 8.3 cm³ for M1 (100%), 11.4 cm³ for M1/2 (65:35%), 12.5 cm³ for M1/2 (35:65%), and 14.3 cm³ for pure M2 (100%). The total volume of the primary MC did not differ between ZA and placebo groups at baseline (12.0 cm³ vs. 10.9 cm³, p=0.55; Table 6). The total volume of the primary MC increased from baseline to one year by 1.6 cm³ in the ZA group vs 2.9 cm³ in the placebo group (p=0.21; Table 6).

The change in M1 volume was larger in the placebo group compared to ZA-group (0.91 cm³ (increase) vs. −0.96 cm³ (decrease); p=0.17). In the ZA-group, the M1 volume decreased by 13% while in the placebo group it increased by 18%. The change in M2 volume was similar in both groups (1.97 cm³ placebo vs. 2.54 cm³ ZA; p=0.62). The M2 volume increased by 56% in the ZA group and by 34% in the placebo group.

Other than primary MC (17 ZA, 8 placebo) were on average 40% smaller in size than the primary MC (6.9 cm³ vs 11.4 cm³) (Table 7). Of them, 15 (88%) were M2-dominant in the ZA group and 6 (75%) in the placebo group. The majority of M2-dominant MC did not convert (12 ZA, 3 placebo) over the follow-up period. The total volume increased by 5% in the ZA group and 11% in the placebo group (Table 7). The proportion of M1 volume of the total volume was 13% in the ZA group and 22% in the placebo group at baseline, and 24% and 36%, respectively, at one year.

### MRI Findings and LBP

Overall change in the primary MC volume did not correlate with the change in intensity of LBP or ODI (Pearson's correlations (r) 0.10 and 0.05, respectively). Within the MC which stayed M1-dominant over the follow-up period, volume change correlated positively with increase in LBP and ODI in the placebo group (r=0.81 and 0.58, respectively) whereas the corresponding correlations were negative and weak in ZA group (r=−0.21 and −0.28, respectively). In the placebo group, within the MC which changed from M2-dominant to M1-dominant the change in the MC volume correlated positively with the change in intensity of LBP and ODI (r=0.70 and 0.89, respectively). The corresponding correlations were negative within the three MC which changed from M1-dominant to M2-dominant (r=−0.72 and −0.98, respectively).

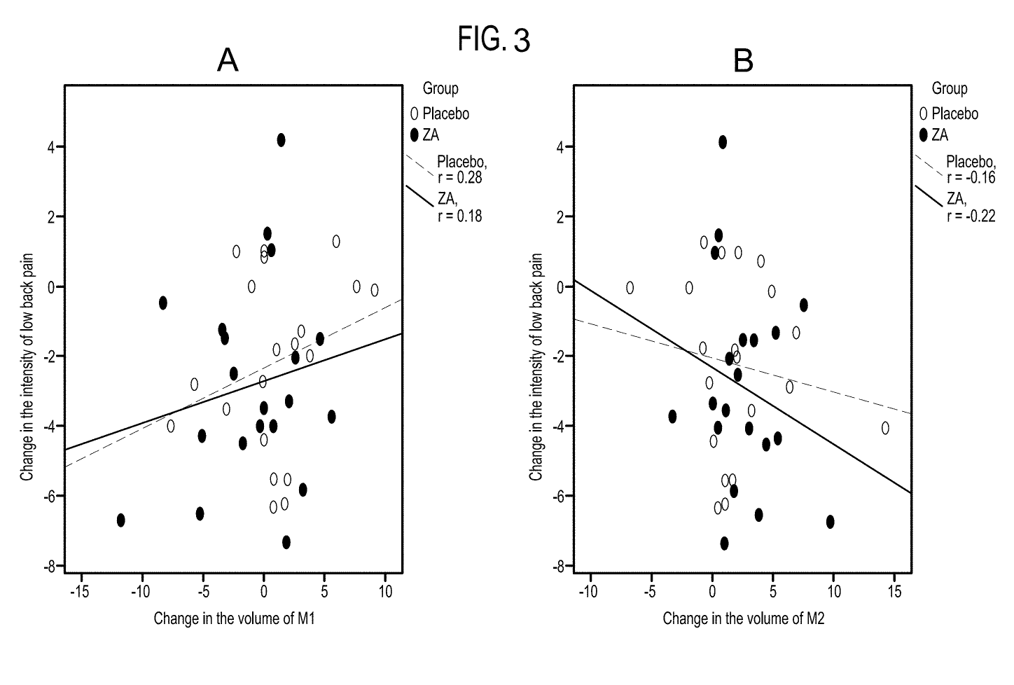

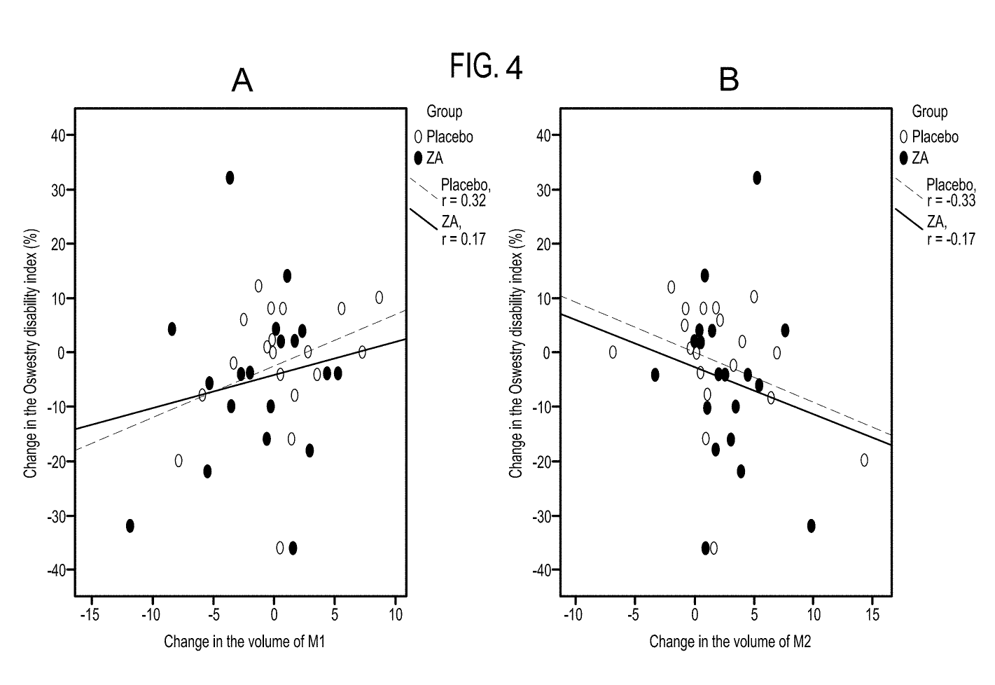

The changes of M1 and M2 volumes in relation to intensity of LBP and ODI are presented in FIG. 3 and FIG. 4. The correlations were weak but consistent with the correlations of MC which stayed either M1- or M2-dominant over follow-up.

To summarize the study results described above, In the ZA group, 85% of patients had M1-dominant primary MC at baseline compared to 50% in the placebo group (p=0.041). The primary MC in the ZA group tended to convert more likely to M2-dominant MC (45% ZA, 15% placebo; p=0.087). Other MC (17 ZA, 8 placebo) were on average 40% smaller in size and remained largely M2-dominant over the one-year period. The M1 volume of the primary MC decreased in the ZA group, whereas it increased in the placebo group (−0.96 cm³ vs 0.91 cm³; p=0.17). The adjusted treatment difference for M1 volume was −1.8 cm³ (95% CI −4.8 to 1.3; p=0.25) and for M2 volume 0.12 cm³ (95% Cl −2.5 to 2.7; p=0.92). The changes in M1 and M2 volumes correlated weakly with changes in intensity of LBP (r=0.24 vs. 0.19, respectively) and ODI (r=0.25 for both).

In the present study, there was a trend of a more likely conversion from M1 to M2 in the ZA group as 45% of M1-dominant MC converted to M2-dominant ones in the ZA group vs. only 15% in the placebo group. Our preliminary hypothesis that ZA might speed up the natural course of MC by enhancing the conversion from M1 to M2, was thus supported by these results. The more likely conversion from M1 to M2 in the ZA group may well be interpreted as a healing process in the natural course of MC. Thus, a single infusion of ZA seemed to influence positively the natural course of MC. In the present study, there was a trend of a more likely conversion from M1 to M2 in the ZA group as 45% of M1-dominant MC converted to M2-dominant ones in the ZA group vs. only 15% in the placebo group. Our preliminary hypothesis that ZA might speed up the natural course of MC by enhancing the conversion from M1 to M2, was thus supported by these results. The more likely conversion from M1 to M2 in the ZA group may well be interpreted as a healing process in the natural course of MC. Thus, a single infusion of ZA seemed to influence positively the natural course of MC.

In the present study, overall change in the MC volume did not correlate with the change in LBP symptoms. In the placebo group, an increase in symptoms for MC-lesions which stayed M1-dominant over the follow-up was observed, whereas in the ZA group the corresponding correlations were negative and weak indicating rather a slight improvement in symptoms.

The present study demonstrated that a single infusion of ZA speeds up the evolution of M1-dominant MC toward M2-dominant and decreases the size of M1. It remains unclear how bisphosphonates influence, but there are several potential mechanisms for bisphosphonates to act. The pathological process of MC is characterized by inflammation, high bone turnover and fibrosis. Chemical and mechanical stimulation of nociceptors adjacent to damaged endplates is probably the source of pain. Growing evidence shows that bisphosphonates exert also effects, in addition to osteoclasts, on osteoblasts, osteocytes and adipocytes, which might explain the positive effects of ZA in this study.

To conclude, this randomized, placebo-controlled, double-blinded trial, MC in the ZA group showed a tendency toward conversion from M1-dominant to M2-dominant and the M1 volume tended to decrease. This study showed that zoledronic acid tended to speed up the conversion of M1-dominant to M2-dominant MC and decrease the volume of M1-dominant MC, which correlated with improvement in symptoms.

TABLE 6
Level and volume of the primary Modic lesion at baseline and follow-up and the change in the volume according to treatment group.

| Volume | Mean (SD) | | | | | Age-adjusted analyses | | Adjusted analyses | |
|---|---|---|---|---|---|---|---|---|---|
| of the primary | ZA | Placebo | Mean (SD) change | | | Difference | | Difference | |
| Modic lesion* | n = 20 | n = 20 | ZA | Placebo | P¹ | (95% CI) | P² | (95% CI) | P³ |
| Level*, n (%) | | | | | | | | | |
| L2/3 | 4 (20) | 0 (0) | | | | | | | |
| L3/4 | 2 (10) | 5 (25) | | | | | | | |
| L4/5 | 7 (35) | 5 (25) | | | | | | | |
| L5/S1 | 7 (35) | 10 (50) | | | | | | | |
| Volume of M1* (cm³) | | | | | | | | | |
| Baseline | 7.44 (4.47) | 5.04 (3.55) | | | | | | | |
| Follow-up | 6.48 (5.25) | 5.95 (4.46) | −0.96 (4.33) | 0.91 (4.02) | 0.17 | −1.92 (−4.65, 0.82) | 0.16 | −1.76 (−4.83, 1.31) | 0.25 |
| Volume of M2* (cm³) | | | | | | | | | |
| Baseline | 4.54 (3.90) | 5.87 (4.84) | | | | | | | |
| Follow-up | 7.09 (4.50) | 7.84 (6.78) | 2.54 (2.94) | 1.97 (4.20) | 0.62 | 0.46 (−1.90, 2.82) | 0.69 | 0.12 (−2.50, 2.75) | 0.92 |
| Total volume* (cm³) | | | | | | | | | |
| Baseline | 11.99 (5.14) | 10.91 (5.96) | | | | | | | |
| Follow-up | 13.57 (5.42) | 13.79 (6.64) | 1.58 (2.18) | 2.88 (3.92) | 0.21 | −1.46 (−3.50, 0.59) | 0.16 | −1.64 (−4.03, 0.76) | 0.17 |

*The primary Modic lesion was assumed to cause patients' symptoms at baseline.
SD = standard deviation,
CI = confidence interval,
ZA = zoledronic acid.
¹Change in the volume compared between the treatment groups, significance from the independent samples t-test.
²Analysis of covariance for change in the volume compared between the treatment groups, adjusted for age.
³Analysis of covariance for change in the volume compared between the treatment groups, adjusted for age, sex, Body Mass Index, and smoking.

TABLE 7
Number, level and volume of other Modic changes (MC) than the primary Modic lesion*
at baseline and follow-up and the change in the volume according to treatment group.

| | Mean (SD) | | | | Unadjusted analyses | |
|---|---|---|---|---|---|---|
| Volume of other than the primary | ZA | Placebo | Mean (SD) change | | Difference | |
| Modic lesion* | n = 17 | n = 8 | ZA | Placebo | (95% CI) | P¹ |
| Multiple MCs, n (%) | | | | | | |
| At two levels | 10 (59) | 6 (75) | | | | |
| At three levels | 7 (41) | 2 (25) | | | | |
| Level of the other than the primary | | | | | | |
| MC, n (%) | | | | | | |
| L1/2 | 1 (5.9) | 0 (0.0) | | | | |
| L2/3 | 1 (5.9) | 1 (12.5) | | | | |
| L3/4 | 3 (17.6) | 0 (0.0) | | | | |
| L4/5 | 5 (29.4) | 4 (50.0) | | | | |
| L5/S1 | 7 (41.2) | 3 (37.5) | | | | |
| Volume of M1* (cm³) | | | | | | |
| Baseline | 1.02 (1.90) | 1.20 (1.62) | | | | |
| Follow-up | 1.88 (2.47) | 2.22 (2.20) | 0.86 (2.19) | 1.03 (2.21) | −0.17 (−2.12, 1.78) | 0.86 |
| Volume of M2* (cm³) | | | | | | |
| Baseline | 6.52 (7.63) | 4.36 (5.27) | | | | |
| Follow-up | 6.05 (6.43) | 3.93 (4.72) | −0.47 (2.44) | −0.43 (1.49) | −0.04 (−1.99, 1.90) | 0.97 |
| Total volume* (cm³) | | | | | | |
| Baseline | 7.53 (7.53) | 5.55 (5.36) | | | | |
| Follow-up | 7.92 (7.31) | 6.15 (5.24) | 0.39 (1.02) | 0.60 (1.53) | −0.21 (−1.28, 0.86) | 0.69 |

*The primary Modic lesion was assumed to cause patients' symptoms at baseline.
SD = standard deviation,
CI = confidence interval,
ZA = zoledronic acid.
¹Independent samples t-test for change in the volume between the treatment groups.

Unless otherwise indicated, all numbers expressing quantities of ingredients, properties such as molecular weight, reaction conditions, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A method of converting a Modic change (MC) comprising: administering a bisphosphonate to a human being, wherein the human being has been selected for suffering from low back pain associated with a Modic change type 1 (M1) or a Modic change that is mixed type 1 and type 2 (M2) with predominating Modic change type 1, wherein the bisphosphonate is administered to:
1) convert a Modic change type 1 to:
a) a Modic change type 2, or
b) a Modic change that is mixed type 1 and type 2, or
2) convert a Modic change that is mixed type 1 and type 2 with predominating Modic change type 1 to:
a) a Modic change type 2, or
b) a Modic change that is mixed type 1 and type 2 with predominating Modic change type 2.

2. The method of claim 1, wherein the bisphosphonate is zoledronic acid.

3. The method of claim 2, wherein the zoledronic acid is in an acid form.

4. The method of claim 2, wherein the zoledronic acid is administered orally.

5. The method of claim 4, wherein the zoledronic acid is in a salt form.

6. The method of claim 5, wherein the zoledronic acid is in a disodium salt form.

7. The method of claim 1, wherein the bisphosphonate speeds up the conversion of M1-dominant to M2-dominant MC.

8. The method of claim 7, wherein the conversion of M1-dominant to M2-dominant MC correlates with improvement in symptoms.

9. The method of claim 1, wherein the bisphosphonate decreases the volume of M1-dominant MC.

10. The method of claim 9, wherein the decrease in the volume of M1-dominant MC correlates with improvement in symptoms.

11. The method of claim 1, wherein the bisphosphonate speeds up the conversion of M1-dominant to M2-dominant MC and decreases the volume of M1-dominant MC.

12. The method of claim 11, wherein the conversion of M1-dominant to M2-dominant MC and the decrease in the volume of M1-dominant MC correlate with improvement in symptoms.

13. The method of claim 2, wherein the zoledronic acid is administered intravenously.

14. The method of claim 13, wherein a single dose of 5 mg of the zoledronic acid is administered.

15. The method of claim 2, wherein about 10 mg to about 300 mg of the zoledronic acid is administered orally.

16. The method of claim 15, wherein a molar equivalent to about 50 mg of the zoledronic acid in the diacid form is administered.

17. The method of claim 16, wherein the zoledronic acid is orally administered weekly.

18. The method of claim 17, wherein the zoledronic acid is orally administered weekly for 6 weeks.

19. The method of claim 1, wherein converting the Modic change results in a reduction in low back pain for the human being.

20. The method of claim 19, wherein the bisphosphonate is the zoledronic acid.

21. The method of claim 20, wherein the zoledronic acid is in an acid form.

22. The method of claim 20, wherein the zoledronic acid is administered orally.

23. The method of claim 22, wherein the zoledronic acid is in a salt form.

24. The method of claim 23, wherein the zoledronic acid is in a disodium salt form.

25. A method of enhancing the natural healing process associated with low back pain, comprising: administering a bisphosphonate to a human being, wherein the human being has been suffering from low back pain associated with a Modic change type 1 or a Modic change that is mixed type 1 and type 2 with predominating Modic change type 1, wherein the bisphosphonate is administered to enhance the natural healing process associated with low back pain.

26. The method of claim 25, wherein the bisphosphonate is zoledronic acid.

27. The method of claim 26, wherein the zoledronic acid is in an acid form.

28. The method of claim 26, wherein the zoledronic acid is administered orally.

29. The method of claim 26, wherein the zoledronic acid is in a salt form.

30. The method of claim 26, wherein the zoledronic acid is in a disodium salt form.
